(19) United States
(12) Patent Application Publication
(10) Pub. No.: US 2005/0256426 A1
(43) Pub. Date: Nov. 17, 2005

(54) Apparatus and method for collecting tissue samples

(76) Inventor: William Brugge, Boston, MA (US)

Correspondence Address: EDWARDS & ANGELL, LLP, P.O. BOX 55874, BOSTON, MA, 02205, US

(21) Appl. No.: 11/128,000

(22) Filed: May 12, 2005

Related U.S. Application Data
(60) Provisional application No. 60/570,158, filed on May 12, 2004.

(51) Int. Cl. A61B 10/00

(52) U.S. Cl. 600/569

(57) ABSTRACT

A tissue sample collection device is disclosed which includes an elongated needle having opposed proximal and distal end portions and having an interior lumen extending therethrough, and a brush accommodated within the interior lumen of the needle for collecting tissue samples, wherein the brush is mounted for movement between a retracted position disposed within the interior lumen of the needle and a protracted position extending from the distal end portion of the needle to collect a tissue sample.

### CROSS-REFERENCE TO RELATED APPLICATION

The subject application claims the benefit of priority from U.S. Provisional Patent Application 60/570,158, filed May 12, 2004, and entitled “Cytology Brush,” the disclosure of which is herein incorporated by reference in its entirety.

### BACKGROUND OF THE INVENTION

1. Field of the Invention

The present invention is directed to a new and useful apparatus for the collection and sampling of tissue cells. Specifically, the present invention is directed to a method and apparatus for collecting tissue samples for analysis in combination with endoscopic ultrasound for analysis of lesions and masses associated with a variety of diseases.

2. Description of the Related Art

Bi opsy is the removal and study of body tissue for medical diagnosis. Typically, physicians obtain biopsy samples in order to detect abnormalities such as cancer and determine the extent to which the disease has spread. To obtain these samples a variety of devices and methods have been suggested for collecting samples from various areas of the body. However, many of the known collection techniques suffer from an inability to repeatably collect sufficient material for analysis or fail to obtain significant material that would be useful in more specifically diagnosing the disease.

One well-known device for collecting tissue samples is the biopsy brush. In one example the brush may be mounted on the end of a catheter. In practice the catheter is inserted into an endoscope which has previously been inserted into the patient and piloted to the point from which a sample is to be taken. After reaching the location of the mass or lesion that the tissue sample is to be taken from, the brush is rubbed across the site to extract cells for later diagnostic analysis. One known device having a brush tipped catheter may be used in conjunction with an ultrasound transducer housed inside the catheter. The ultrasound transducer allows the physician to view the area in greater detail than other endoscopic viewing methods, including the ability to view layers of tissue at varying depths.

Another device using a biopsy brush uses a dual or multi-lumen endoscope having the brush carried inside one of the lumens. The brush is typically located at the end of a stylet allowing the physician to operate the brush independent of the endoscope. The physician places the endoscope in the area where a sample is to be taken, and then inserts the stylet into the lumen of the endoscope, upon reaching the end of the endoscope, the brush is allowed to exist the endoscope and brush against or debride an area from which a tissue sample is to be taken. After completion of the debriding step, the physician removes the stylet and brush from the endoscope for analysis. Often multiple brushes are used to collect samples from a single lesion or mass of interest.

Yet another known method for the collection of tissue samples is referred to as fine needle aspiration (FNA). The device once again uses an endoscope in connection with the fine needle arrangement, and in some devices may also be used in conjunction with ultrasound techniques. During FNA, the lesion or mass is identified using known imaging/sighting methods, such as ultrasound. The needle is extended through the endoscope and inserted into the mass or lesion. Alternatively fluid may be injected into the site and then suctioned out or just suction may be applied to the site following insertion of the needle to collect a tissue sample. This technique has been particularly practiced in combination with endoscopic ultrasound to accurately identify the areas to be sampled and to pilot the needle to the proper location and depth.

However, each of these methods suffers from certain drawbacks. For example, the brush mounted on a stylet or catheter can only take samples from the exterior of a mass or lesion and would not be able to penetrate a tissue layer to reach a tumor or mass located on the exterior wall of the intestine or lung, for example. Similarly, while FNA has proven to be a very effective diagnostic tool, it often fails to detect certain tissue fragments that may be useful to more thorough diagnosis.

Accordingly, the present invention is directed to overcoming these and other shortcomings of the prior art devices.

### SUMMARY OF THE INVENTION

The subject invention is directed to a new and useful device for collecting tissue samples. The device includes an elongated needle having opposed proximal and distal end portions and an interior lumen, which extends therethrough. The device further includes a brush that is accommodated within the interior lumen of the needle for collecting tissue samples. The brush is mounted for movement between a retracted position disposed within the interior lumen of the needle and a protracted position extending from the distal end portion of the needle to collect a tissue sample. Preferably, the device further includes means for facilitating movement of the brush relative to the needle between a retracted position and a protracted position.

The distal end portion of the needle is preferably adapted and configured for puncturing tissue. Accordingly, a beveled cutting edge is defined at the distal end portion of the needle. In an embodiment of the invention, the brush includes bristles that extend generally perpendicular to a longitudinal axis thereof. In another embodiment of the invention, the brush includes bristles that extend at an angle to a longitudinal axis thereof. It is envisioned that the bristles of the brush may be formed at least in part from metal or plastic.

In an embodiment of the invention, a multi-lumen catheter is provided, wherein an ultrasound transducer is housed in a first lumen of the multi-lumen catheter for imaging tissue to be sampled and the needle is accommodated within a second lumen of the multi-lumen catheter for obtaining a sample of the tissue using the brush. In such an instance, the needle is mounted for movement within the second lumen between a retracted position and a protracted position extending from a distal end of the catheter.

The subject invention is also directed to a new and useful method of extracting a tissue sample. The method includes the steps of providing an elongated needle having an interior lumen extending therethrough for accommodating a brush configured to collect a tissue sample, inserting a distal end portion of the needle into an area of tissue from which a sample is desired, extending the brush from the distal end of the needle and into the tissue from which a sample is desired, retracting the brush from the tissue and into the needle and then retracting the needle from the tissue. Preferably, the method further includes the step of reciprocating the brush in tissue from which a sample is desired.

The method also includes the step of providing an ultrasound transducer for imaging an area of tissue to determine where to insert the needle and collect the tissue sample. Accordingly, the method includes the step of providing a multiple lumen catheter having a first lumen for accommodating the ultrasound transducer and a second lumen for accommodating the needle. The method further includes the step of withdrawing the needle from the catheter following retraction of the brush and extraction of the needle from the tissue. Also includes in the method is the step of inserting at least a second needle into the catheter to extract a second sample.

The subject invention is also directed to an apparatus for extracting a tissue sample from tissue that includes, among other things, a flexible catheter having at least one lumen extending therethrough, and an elongated needle accommodated with the lumen of the catheter and having an interior lumen extending therethrough, and a brush accommodated within the interior lumen of the needle for collecting tissue samples.

These and other aspects of the apparatus and method of the subject invention will become more readily apparent to those having ordinary skill in the art from the following detailed description of the invention taken in conjunction with the drawings.

### BRIEF DESCRIPTION OF THE DRAWINGS

So that those having ordinary skill in the art to which the present invention pertains will more readily understand how to employ the novel apparatus and method of the present invention, embodiments thereof will be described in detail hereinbelow with reference to the drawings, wherein:

FIG. 1 is perspective view of a tissue-sampling device constructed in accordance with a preferred embodiment of the subject invention, which includes an elongated needle and sampling brush movably accommodated within the needle;

FIG. 1A is a perspective view of a distal end portion of the needle of FIG. 1 and a sampling brush that has perpendicular bristles;

FIG. 1B is a perspective view of a distal end portion of the needle of FIG. 1 and a sampling brush that has angled bristles;

FIG. 2 is an illustration of the tissue-sampling device of the subject invention being employed in conjunction with an endoscopic ultrasound catheter according to the present invention;

FIG. 3 is a perspective view of an endoscope configured for use in conjunction with the tissue-sampling device of the subject invention;

FIG. 4A is an enlarged side elevational view of the distal end portion of an imaging endoscope, illustrating the tissue-sampling device of the subject invention in a retracted position against the bias of an actuation spring;

FIG. 4B shows the tissue-sampling device of the subject invention in an extended operational position after the spring has been released from the position shown in FIG. 4A; and

FIG. 5 is an enlarged side elevational view of the distal end portion of the endoscope of FIG. 3, in partial cross-section, illustrating the tissue-sampling device of the subject invention in a deployed position to obtain a tissue sample from a target mass.

### DETAILED DESCRIPTION OF PREFERRED EMBODIMENTS

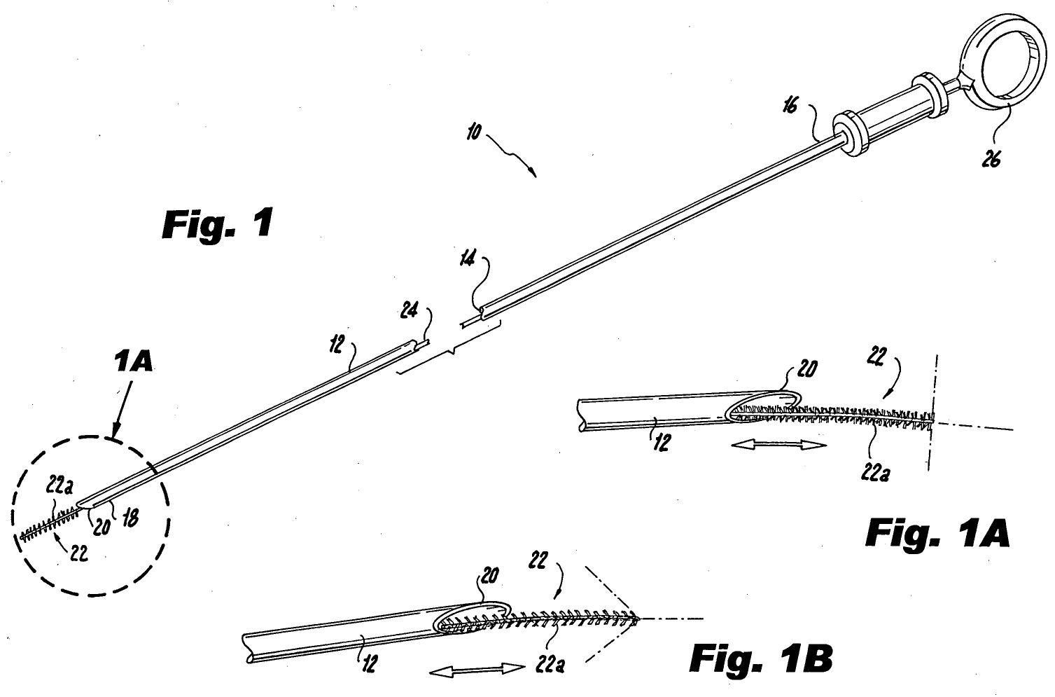

Referring now-to the drawings wherein like reference numerals identify similar aspects or features of the subject invention, there is illustrated in FIG. 1, a tissue sampling apparatus or device constructed in accordance with a preferred embodiment of the subject invention and designated generally by reference numeral 10. The tissue sampling device 10 of the subject invention includes an elongated surgical needle 12 having a central lumen 14 extending therethrough which extends from a proximal end portion 16 of the needle 12 to a distal end portion 18 of the needle 12. The distal end portion 18 of needle 12 is sharpened or otherwise formed with a tapered bevel defining at least one angularly biased cutting edge 20 for puncturing tissue.

In an embodiment of the invention, the needle 12 is configured as an aspiration needle designed to obtain cytologic materials from gastro-intestinal tumors or the like through the use of suction. For example, the aspiration needle may be designed for use in fine needle aspiration (FNA) biopsy. It is envisioned that needle 12 has standard dimensions for performing FNA biopsy. For example, the needle can be a fine gauge needle such as a 19 gauge needle. Also, needle 12 can be formed from conventional needle fabrication materials, such as, for example, stainless steel. The needle may be primarily rigid or flexible in nature, depending upon its intended manner of use and deployment.

A tissue collection brush 22 is housed or otherwise accommodated within the central lumen 14 of needle 12 and it is mounted for movement between a retracted position disposed within the interior lumen of the needle and a protracted position extending from the distal end portion of the needle to collect a tissue sample. The tissue collection brush 22 is preferably elongated in configuration, however the specific shape can vary. Preferably, the brush 22 includes a multiplicity of bristles 22a. In one embodiment shown in FIG. 1A, the bristles 22a extend outwardly from the center of the brush 22 in a generally perpendicular manner from the longitudinal axis thereof. Alternatively, the brush 22 includes bristles 22a that extend at an angle to the longitudinal axis thereof, as shown in FIG. 1B. It is envisioned that the bristles 22a of brush 22 are sufficiently rigid to obtain a tissue sample and are preferably made of a biocompatible material, such as, for example a metal material or a plastic material.

In an embodiment of the subject invention, the brush 22 is operatively associated with or otherwise connected to an elongated stylet 24, which allows a physician to move the brush 22 relative to the needle 12, for example, to extend the brush 22 from the distal end portion 18 of the needle 12 to collect a sample and to subsequently retract the brush 22 into the central lumen 14 of the needle 12 after the tissue sample has been collected, as best seen in FIG. 2. A manual actuator 26 is operatively associated with the proximal end portion 16 of needle 12 for moving the stylet 24 between extended and protracted positions, or for otherwise reciprocating the brush 22.

The sample collection brush and needle assembly of the subject invention is preferably operatively associated with a conventional FNA handle sheath and plunger assembly or a syringe assembly, as is currently known in the art. In conjunction therewith, the brush 22 in inserted into the needle 12 using a stylet 24. The stylet 24 may be a rigid longitudinal shaft. However, the rigidity of the shaft need only be sufficient to accommodate the deployment of the brush from the needle. Indeed, the shaft may be somewhat flexible. Alternatively, the stylet 24 associated with sampling brush 22 may be formed from braided or otherwise twisted wires or another design known to those of skill in the art.

In accordance with an embodiment of the subject invention, the tissue sampling device 10, with its needle 12 and sampling brush 22 is readily employed in conjunction with a surgical deployment catheter, and more particularly, with an endoscopic ultrasound catheter 50 (see FIG. 2), for analysis of lesions and masses associated with a variety of diseases. Thus, in operation, a catheter, endoscope or endosonoscope 50 is piloted to the area of interest, as shown for example, in FIG. 2. This is typically a mass or lesion from which a sample is to be taken using tissue sampling device 10. Examples of endoscopes with which the present invention may be employed include, but are not limited to the Olympus mechanical puncture echoendoscope (Olympus GM UM 30P).

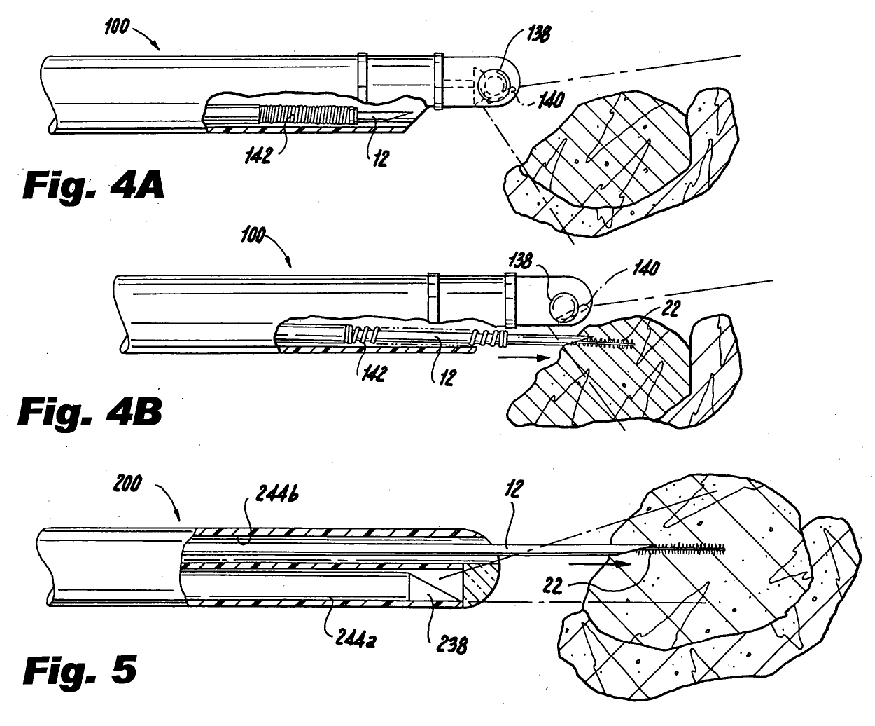

Referring to FIG. 4A and 4B, there is depicted the distal end portion of such an endoscope which is designated generally by reference numeral 100. As shown, endoscope 100 includes a stationary transducer 138, which generates an ultrasound signal, and a mirror 140, which directs the ultrasound signal in such a manner so that it is focused on a desired tissue location. Endoscope 100 is also provided with the subject needle 12, which is housed in the endoscope until deployment. As shown, needle 12 includes a readily deployable biasing spring 142. The spring 142 offers mechanical assistance in puncturing tissue and membrane layers to take samples therefrom.

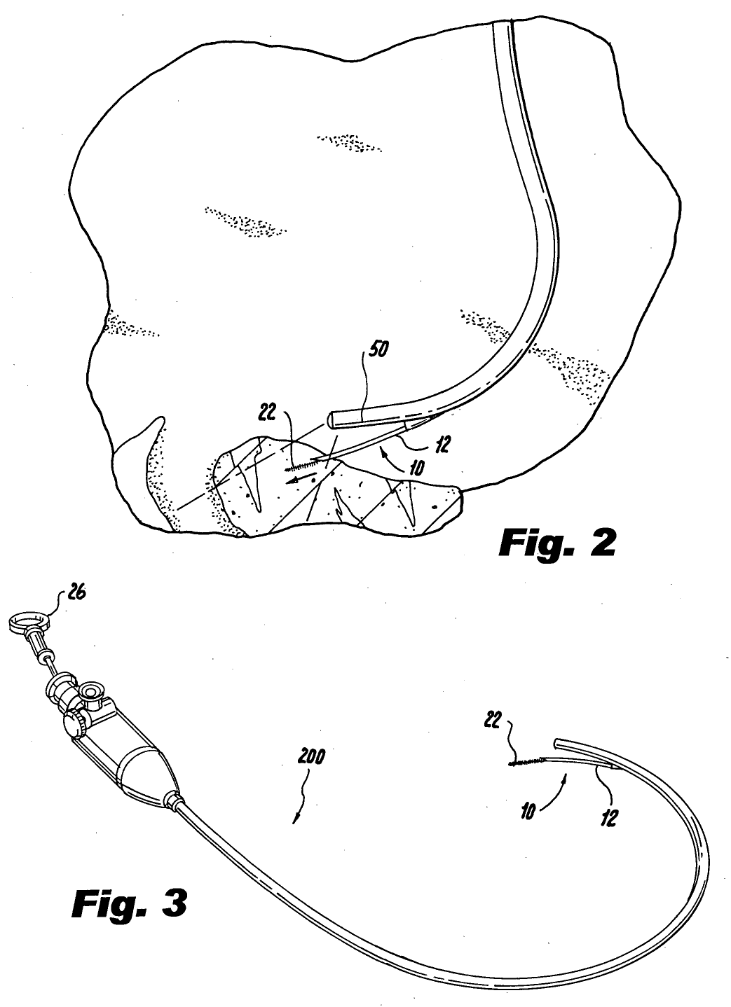

Alternatively, a more traditional endoscope, such as an Olympus Curved Linear Array Endoscope, which has been commonly used for FNA, may be used by the medical professional, in conjunction with the tissue-sampling device of the subject invention. Such an endoscope is shown in FIGS. 3 and 5, and is designated generally by reference numeral 200. As shown in FIG. 5, the multi-lumen endoscope 200 includes a transducer 238, which is housed in lumen 244a of endoscope 200, and the device 10 with its combined needle 12 and brush 22 is housed in lumen 224b of endoscope 200.

Unlike the mechanical puncturing endoscope 100 discussed above with reference to FIGS. 4A and 4B, in this instance, needle 12 is configured to be manually driven by a stylet (see FIG. 3), which extends through the lumen 244b and terminates at an actuation handle at or near the proximal end of the endoscope 200 to allow the physician to extend or retract the needle 12 as desired. Naturally, the physician can extend and retract the brush 22 from the needle 12 through the use of another stylet which also has a handle or actuator located at or near the proximal end portion of endoscope 200 (see FIG. 3).

Once the endoscope 200 is positioned at or near the area from which a sample is to be taken, the needle 12 is deployed, as shown in FIG. 5. With use of ultrasound imaging, the needle 12 can be more precisely positioned for entry into the tissue to be sampled.

When using endoscope 100, which includes mechanical spring puncture assistance, once the distal end of the needle 12 is extended from the endoscope 100 adjacent the tissue to be sampled, as shown in FIG. 4A, the spring 142 is released from a biased position. At such a time, the needle 12 is propelled forward under the force of the spring 142 into the mass or lesion, as shown in FIG. 4B.

Alternatively, as shown in FIG. 5, when using the more traditional endoscope 200, the physician uses a stylet to extend the needle 12 from the lumen 244b within which it is accommodated, and insert it into the mass or lesion to be sampled. Upon entry of the needle 12 into the tissue to be sampled, the physician can deploy the brush 22. The brush 22 is moved back and forth with handle 26 within the tissue to ensure that a sufficient number of cells are trapped on the bristles 22a of the brush 22. The brush 22 can then be retracted into the needle 12, the needle 12 may then be retraced from the endoscope 200, and the tissue sample may then be prepared for analysis. As with FNA procedures, it is often desirable to take several samples from a single mass or lesion. Accordingly, the physician may insert one or more other needles into the endoscope and collect as many additional samples as desired.

As will be appreciated by those of skill in the art, although not the preferred embodiment, the cytology brush of the present invention may also be used to take samples from the exterior of masses and lesions without puncturing the tissue without departing from the scope of the present invention.

### EXAMPLE

An experiment was conducted on eleven patients previously referred for electronic ultrasound-FNA (EUS-FNA). A side-by-side comparison was made of the EUS-FNA and the biopsy brush of the present invention. The masses were first aspirated in a standard fashion using two passes of a 22 g FNA needle (Wilson Cook). Following the FNA, a 19 g needle containing the brush device was placed in the endoscope and deployed into the mass. The brush was gently agitated within the mass to obtain cellular material.

The FNA materials were then analyzed by direct smear and the brush biopsy samples were rinsed in saline and processed using either ThinPrep or a cellblock. FNA was diagnostic in all eleven patients while the biopsy brush was diagnostic in seven of the eleven patients. However, the biopsy brush produced architecturally important tissue fragments that enhanced cytological diagnosis in three of the eleven cases that were not produced by the FNA analysis.

Disparity in the results may be attributable to a number of factors including the familiarity of the physicians in using the device, the different analytical techniques, and the potential for optimization of the biopsy brush itself. It is envisioned and well within the scope of the subject disclosure that the design or configuration of the tissue sampling brush 22 can be optimized to achieve better results such as by altering the angle and direction of the bristles 22a on the brush 22 in relation to the stylet 24, and maximizing the opening size and direction or biased edge 20 at the opening in the distal end portion 18 of the needle 12 to prevent the removal of sample material from the bristles 22a as the brush 22 is retracted from the mass into the needle lumen 14.

Because of the significant tissue samples resulting from the experiments, and the ease at which the biopsy brush is alternated with the FNA tools, complimentary use is seen as an immediate benefit of the present invention resulting in more specific diagnosis than FNA alone.

Although the apparatus and method of the subject invention have been described with respect to preferred embodiments, those skilled in the art will readily appreciate that changes and modifications may be made thereto without departing from the spirit and scope of the subject invention as defined by the appended claims.

1. A tissue sample collection device comprising:
a) an elongated needle having opposed proximal and distal end portions and having an interior lumen extending therethrough; and
b) a brush accommodated within the interior lumen of the needle for collecting tissue samples, wherein the brush is mounted for movement between a retracted position disposed within the interior lumen of the needle and a protracted position extending from the distal end portion of the needle to collect a tissue sample.

2. A tissue sample collection device as recited in claim 1, further comprising means for facilitating movement of the brush relative to the needle between a retracted position and a protracted position.

3. A tissue sample collection device as recited in claim 1, wherein the distal end portion of the needle is adapted and configured for puncturing tissue.

4. A tissue sample collection device as recited in claim 3, wherein a beveled cutting edge is defined at the distal end portion of the needle.

5. A tissue sample collection device as recited in claim 1, wherein the brush includes bristles extending generally perpendicular to a longitudinal axis thereof.

6. A tissue sample collection device as recited in claim 1, wherein the brush includes bristles extending at an angle to a longitudinal axis thereof.

7. A tissue sample collection device as recited in claim 1, wherein the brush includes bristles formed at least in part from metal.

8. A tissue sample collection device as recited in claim 1, wherein the brush includes bristles formed at least in part from plastic.

9. A tissue sample collection device as recited in claim 1, wherein the needle is operatively associated with a catheter.

10. A tissue sample collection device as recited in claim 1, wherein the needle is operatively associated with an endoscope.

11. A tissue sample collection device as recited in claim 1, wherein the needle is operatively associated with an endoscopic ultrasound catheter.

12. A tissue sample collection device as recited in claim 1, further comprising a multi-lumen catheter, wherein an ultrasound transducer is housed in a first lumen of the multi-lumen catheter for imaging tissue to be sampled and said needle is accommodated within a second lumen of said multi-lumen catheter for obtaining a sample of the tissue.

13. A tissue sample collection device as recited in claim 12, and wherein the needle is o mounted for movement within the second lumen between a retracted position and a protracted position extending from a distal end of the catheter.

14. A method of extracting a tissue sample comprising the steps of:
a) providing an elongated needle having an interior lumen extending therethrough for accommodating a brush configured to collect a tissue sample;
b) inserting a distal end portion of the needle into an area of tissue from which a sample is desired;
c) extending the brush from the distal end of the needle and into the tissue from which a sample is desired;
d) retracting the brush from the tissue and into the needle; and
e) retracting the needle from the tissue.

15. A method according to claim 14 further comprising the step of reciprocating the brush in tissue from which a sample is desired.

16. A method according to claim 14, further comprising the steps of providing an ultrasound transducer for imaging an area of tissue to determine where to insert the needle and collect the tissue sample.

17. A method according to claim 16, further comprising the step of providing a multiple lumen catheter having a first lumen for accommodating the ultrasound transducer and a second lumen for accommodating the needle.

18. A method according to claim 17, further comprising the step of withdrawing the needle from the catheter following retraction of the brush and extraction of the needle from the tissue.

19. A method according to claim 18, further comprising the step of inserting at least a second needle into that catheter to extract a second sample.

20. Apparatus for extracting a tissue sample from tissue comprising:
a) a catheter having at least one lumen extending therethrough;
b) an elongated needle accommodated with the lumen of the catheter and having an interior lumen extending therethrough; and
c) a brush accommodated within the interior lumen of the needle for collecting tissue samples.
